(19) United States
(12) Patent Application Publication
(10) Pub. No.: US 2024/0307038 A1
(43) Pub. Date: Sep. 19, 2024

(54) PROBE CONTROL APPARATUS, NON-TRANSITORY COMPUTER READABLE MEDIUM, AND ULTRASOUND DIAGNOSTIC APPARATUS

(71) Applicant: Canon Medical Systems Corporation, Otawara-shi (JP)

(72) Inventors: Kaisei ONO, Nasushiobara (JP); Akihiro KAKEE, Nasushiobara (JP)

(73) Assignee: Canon Medical Systems Corporation, Otawara-shi (JP)

(21) Appl. No.: 18/604,837

(22) Filed: Mar. 14, 2024

(30) Foreign Application Priority Data
Mar. 14, 2023 (JP) 2023-039648

(51) Int. Cl. A61B 8/00

(52) U.S. Cl. CPC A61B 8/54; A61B 8/4444; A61B 8/56

(57) ABSTRACT

According to one embodiment, a probe control apparatus includes processing circuitry. With respect to a scanning line in which a change has been made to a depth of field, the processing circuitry makes a change to a pulse repetition frequency in accordance with the depth of field after the change. The processing circuitry sets a new transmission voltage based on the pulse repetition frequency after the change.

### CROSS-REFERENCE TO RELATED APPLICATIONS

This application is based upon and claims the benefit of priority from Japanese Patent Application No. 2023-039648, filed Mar. 14, 2023, the entire contents of which are incorporated herein by reference.

### FIELD

Embodiments described herein relate generally to a probe control apparatus, a non-transitory computer readable medium, and an ultrasound diagnostic apparatus.

### BACKGROUND

There exists a radial probe configured to display an image in an annular shape by performing a 360-degree scan. With such a radial probe, in the case where observation of a deeper region of interest in a living body is desired, in general, a depth of field of the entire annular shape is deepened. This also deepens a depth of field of regions other than the region of interest, thereby resulting in a decreased frame rate.

Considering this, there exists a method by which only a region of interest can be observed with a deep depth of field without decreasing a frame rate by controlling driving of ultrasound vibrators of the radial probe based on a position of each vibrator and a distance to a drawing area. However, with a transmission voltage being constant between scanning lines, in the case of setting the transmission voltage in accordance with a region having a deep depth of field, a greater-than-necessary transmission voltage is applied to a region having a shallow depth of field, thereby leading to the possibility that heat exceeding an allowable value may be generated. On the other hand, in the case of setting a transmission voltage in accordance with a region having a shallow field of view, there is a problem wherein a transmission power that can reach the region having a deep depth of field cannot be supplied and sensitivity is decreased.

### BRIEF DESCRIPTION OF THE DRAWINGS

FIG. 1 is a block diagram showing a configuration example of an ultrasound diagnostic apparatus according to a present embodiment.

FIG. 2 is a diagram showing a shape example of an ultrasound probe according to the present embodiment.

FIG. 3 is a flowchart for illustrating an operational sequence of a probe control apparatus according to the present embodiment.

FIG. 4 is a conceptual diagram illustrating a change state of a depth of field according to an embodiment.

### DETAILED DESCRIPTION

In general, according to one embodiment, a probe control apparatus includes processing circuitry. With respect to a scanning line in which a change has been made to a depth of field, the processing circuitry makes a change to a pulse repetition frequency in accordance with the depth of field after the change. The processing circuitry sets a new transmission voltage based on the pulse repetition frequency after the change.

A probe control apparatus according to a present embodiment includes a change unit and a setting unit. With respect to a scanning line in which a change has been made to a depth of field, the change unit makes a change to a pulse repetition frequency in accordance with the depth of field after the change. The setting unit sets a new transmission voltage based on the pulse repetition frequency after the change) .

Hereinafter, a probe control apparatus, a non-transitory computer readable medium, and an ultrasound diagnostic apparatus according to the present embodiment will be described with reference to the drawings. In the following embodiments, elements assigned the same reference numeral perform the same operation, and redundant descriptions will be omitted as appropriate.

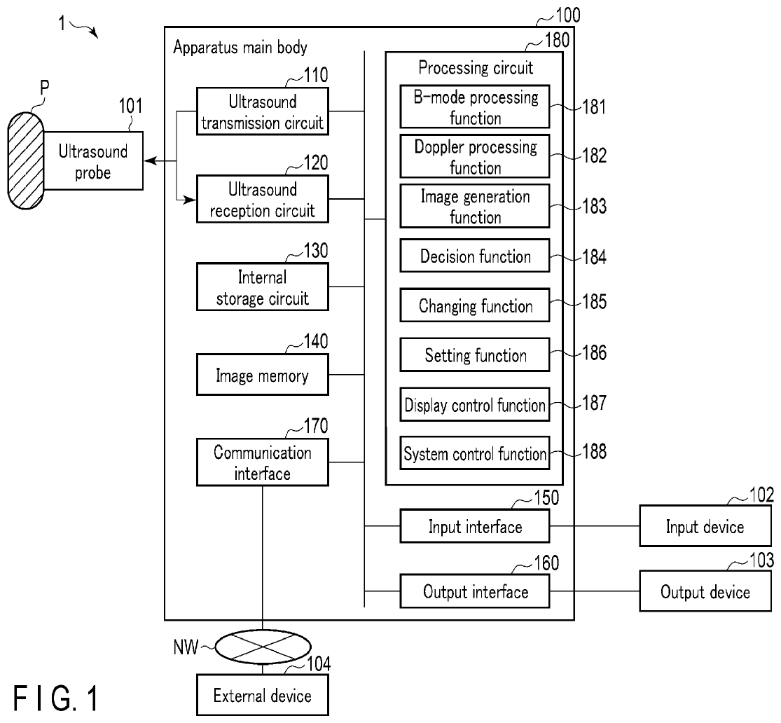

FIG. 1 is a diagram showing a configuration example of the ultrasound diagnostic apparatus according to the present embodiment. An ultrasound diagnostic apparatus 1 shown in FIG. 1 includes an apparatus main body 100 including a probe control apparatus, and an ultrasound probe 101. The apparatus main body 100 is connected to an input device 102 and an output device 103. Furthermore, the apparatus main body 100 is connected to an external device 104 via a network NW. Examples of the external device 104 include a server equipped with a picture archiving and communication systems (PACS), a workstation capable of performing post-processing, etc.

The ultrasound probe 101 performs ultrasound scanning on a scan region in a living body P, which is a subject, under a control of the apparatus main body 100. The ultrasound probe 101 includes, for example, an acoustic lens, one or more matching layers, a plurality of vibrators (piezoelectric elements), a backing member, etc. The acoustic lens is formed of, for example, silicon rubber, and converges ultrasound beams. The one or more matching layers perform impedance matching between the plurality of vibrators and a living organism. The backing member prevents backward propagation of ultrasound waves from the plurality of vibrators in relation to a radiant direction. It is assumed that the ultrasound probe 101 is, for example, a radial probe capable of performing a 360-degree scan; however, the ultrasound probe 101 may be a linear probe or a convex probe. The ultrasound probe 101 is detachably connected to the apparatus main body 100. The ultrasound probe 101 may be provided with a button which is to be pressed at the time of offset processing, ultrasound image-freezing operation (freeze operation), etc.

The plurality of vibrators generate an ultrasound wave based on a drive signal supplied from an ultrasound transmission circuit 110 described later, included in the apparatus main body 100. Accordingly, an ultrasound wave is transmitted from the ultrasound probe 101 to the living body P. In response to an ultrasound wave being transmitted from the ultrasound probe 101 to the living body P, the transmitted ultrasound wave is repeatedly reflected by the discontinuous acoustic-impedance surfaces of the body tissues in the living body P, thereby being received as echo signals by the piezoelectric vibrators. The amplitudes of the echo signals to be received vary depending on the difference in acoustic impedance between the discontinuous surfaces that have reflected the ultrasound waves. Furthermore, in the case of the transmitted ultrasonic pulse being reflected from the surface of, for example, a moving bloodstream or a cardiac wall, the echo signals receive a frequency shift due to the Doppler effect, depending on the velocity component in the ultrasound transmission direction of a moving object. The ultrasound probe 101 receives the echo signals from the living body P and converts the echo signals into electric signals.

FIG. 1 illustrates a connection relationship between the ultrasound probe 101 and the apparatus main body 100. However, a plurality of ultrasound probes may be connected to the apparatus main body 100. Which one of the connected ultrasound probes is to be used for the ultrasound scanning can be selected freely through, for example, a software button on a touch panel (to be described later).

The apparatus main body 100 is an apparatus configured to generate ultrasound images based on the echo signals (referred to as echo signals) received by the ultrasound probe 101. The apparatus main body 100 includes the ultrasound transmission circuit 110, an ultrasound reception circuit 120, an internal storage circuit 130, an image memory 140, an input interface 150, an output interface 160, a communication interface 170, and a processing circuitry 180. The processing circuitry 180 is also referred to as a probe control apparatus.

The ultrasound transmission circuit 110 is a processor for supplying drive signals to the ultrasound probe 101. The ultrasound transmission circuit 110 is realized by, for example, a trigger generating circuit, a delay circuit, a pulser circuit, etc. The trigger generating circuit repeatedly generates a rate pulse for forming ultrasound waves for transmission, at a predetermined rate frequency. The delay circuit applies a delay time to each rate pulse generated by the trigger generating circuit. Herein, the delay time is intended for each of the piezoelectric vibrators, and is required for converging the ultrasound waves output from the ultrasound probe into a beam shape and determining the transmission directivity. The pulser circuit applies the drive signals (drive pulses) to the plurality of ultrasound vibrators provided in the ultrasound probe 101 at timings according to the rate pulses. By the delay circuit varying the delay time applied to each rate pulse, the direction of transmission from the surfaces of the piezoelectric vibrators can be discretionarily adjusted.

The ultrasound transmission circuit 110 is also capable of discretionarily changing the output intensity of ultrasound waves using the drive signals. By increasing output intensity, the ultrasound diagnostic apparatus can suppress the influence of attenuation of the ultrasound waves within the living body P. At the time of reception, the ultrasound diagnostic apparatus can acquire an echo signal with a high signal-noise ratio (SNR) by reducing the influence of attenuation of ultrasound waves.

Generally, in response to an ultrasound wave propagating within the living body P, the intensity of ultrasonic vibrations (also called “sound power”) corresponding to the output intensity is attenuated. The attenuation of sound power is caused by absorption, scattering, reflection, etc. The degree of attenuation of the sound power depends on the frequency of the ultrasound wave and the distance in the radial direction of the ultrasound wave. For example, the degree of attenuation is increased by increasing the frequency of the ultrasound wave. Furthermore, the degree of attenuation is increased as the distance in the radial direction of the ultrasound wave becomes longer.

The ultrasound reception circuit 120 is a processor that performs various types of processing on the echo signal received by the ultrasound probe 101 and thereby generates a reception signal. The ultrasound reception circuit 120 generates a reception signal with respect to the echo signal of the ultrasound wave acquired by the ultrasound probe 101. Specifically, the ultrasound reception circuit 120 is realized by, for example, a preamplifier, an A/D converter, a demodulator, a beam former (adder), etc. The preamplifier performs gain correction processing by amplifying the echo signal received by the ultrasound probe 101 for each channel. The A/D converter converts the gain-corrected echo signal into a digital signal. The demodulator demodulates the digital signal. The beam former provides the demodulated digital signal with the delay time required to determine the reception directivity, and adds the digital signals to which the delay time is provided. Through the addition processing by the beam former, a reception signal in which a reflected component in a direction corresponding to the reception directivity is enhanced is generated. The reception signal may be referred to as an IQ signal. Furthermore, the ultrasound reception circuit 120 may store the reception signal (IQ signal) in the internal storage circuit 130 described later, or may output it to the external device 104 via the communication interface 170.

The internal storage circuit 130 includes a processor-readable storage medium, such as a magnetic storage medium, an optical storage medium, or a semiconductor memory. The internal storage circuit 130 stores therein a program and various data, etc., for realizing ultrasound transmission/reception. The programs and various data may be pre-stored in the internal storage circuit 130. Alternatively, the programs and various data may be stored and distributed in a non-transitory storage medium, read from the non-transitory storage medium, and installed in the internal storage circuit 130. In response to operational inputs given via the input interface 150, the internal storage circuit 130 stores, e.g., B-mode image data and contrast image data generated by the processing circuitry 180, and image data relating to bloodstream visualization generated by the processing circuitry 180. The internal storage circuit 130 can transfer the stored image data to the external device 104 or the like via the communication interface 170. The internal storage circuit 130 may store the reception signal (IQ signal) generated by the ultrasound reception circuit 120, or may transfer it to the external device 104 or the like via the communication interface 170.

The internal storage circuit 130 may be, e.g., a drive device which reads and writes various types of information to and from a portable storage medium, such as a CD-ROM drive, a DVD drive, and a flash memory. The internal storage circuit 130 may write the stored data onto a portable storage medium to store the data into the external device 104 by way of the portable storage medium.

The image memory 140 includes, for example, a processor-readable storage medium such as a magnetic or optical storage medium, a semiconductor memory, etc. The image memory 140 stores image data items corresponding to a plurality of frames immediately before a freeze operation input via the input interface 150. The image data stored in the image memory 140 is, for example, continuously displayed (cine-displayed).

The internal storage circuit 130 and the image memory 140 are not necessarily implemented by independent storage devices. The internal storage circuit 130 and the image memory 140 may be implemented by a single storage device. Each of the internal storage circuit 130 and the image memory 140 may be implemented by a plurality of storage devices.

The input interface 150 receives various instructions from an operator through the input device 102. The input device 102 is, for example, a mouse, a keyboard, a panel switch, a slider switch, a trackball, a rotary encoder, an operation panel, or a touch command screen (TCS). The input interface 150 is coupled to the processing circuitry 180 via a bus, for example, so that it can convert an operation instruction that is input by the operator into an electric signal, and output the electric signal to the processing circuitry 180. The input interface 150 is not limited to physical operation components such as a mouse and a keyboard. Examples of the input interface 150 also include a circuit which receives an electric signal corresponding to an operational instruction input from an external input device separate from the ultrasound diagnostic apparatus 1, and outputs the electric signal to the processing circuitry 180.

The output interface 160 is an interface to output, for example, the electric signal from the processing circuitry 180 to the output device 103. The output device 103 may be any discretionarily employed display such as a liquid crystal display, an organic EL display, an LED display, a plasma display, a CRT display, etc. The output device 103 may be a touch-panel display that also serves as the input device 102. The output device 103 may also include a speaker configured to output a voice in addition to the display. The output interface 160 is connected to the processing circuitry 180, for example, via a bus, and outputs the electric signal from the processing circuitry 180 to the output device 103.

The communication interface 170 is connected to the external device 104 via, for example, the network NW so that it performs data communication with the external device 104.

The processing circuitry 180 is, for example, a processor functioning as a center of the ultrasound diagnostic apparatus 1. The processing circuitry 180 executes the programs stored in the internal storage circuit 130, thereby realizing the functions corresponding to the programs. The processing circuitry 180 has, for example, a B-mode processing function 181, a Doppler processing function 182, an image generation function 183, a decision function 184, a change function 185, a setting function 186, a display control function 187, and a system control function 188.

The B-mode processing function 181 is a function of generating B-mode data based on the reception signal received from the ultrasound reception circuit 120. In the B-mode processing function 181, the processing circuitry 180 performs envelope detection processing, logarithmic compression processing, etc., on the reception signal received from the ultrasound reception circuit 120, for example, to generate data (B-mode data) that expresses signal intensity by brightness. The generated B-mode data is stored in a raw data memory (not shown in the drawings) as B-mode raw data on a two-dimensional ultrasound scanning line (raster).

The Doppler processing function 182 is a function of generating, by analyzing the frequencies of the reception signals received from the ultrasound reception circuit 120, data information (Doppler information) obtained by extracting motion information of a moving object in a region of interest (ROI) that is set in a scan area, based on the Doppler effect. The generated Doppler information is stored in a raw data memory (not shown in the drawings) as Doppler raw data (also called Doppler data) on a two-dimensional ultrasound scanning line.

Specifically, by the Doppler processing function 182, the processing circuitry 180 estimates, as the motion information of the moving object, an average velocity, an average dispersion value, an average power value, etc. at each of the sampling positions, and generates Doppler data indicating the estimated motion information. The moving object is, for example, a bloodstream, tissue portions such as the cardiac wall, a contrast medium, etc. The processing circuitry 180 estimates, by the Doppler processing function 182, an average bloodstream velocity, a dispersion value of the bloodstream velocity, and a power value of a bloodstream signal as motion information of the bloodstream (bloodstream information) at each of the sampling positions, and generates Doppler data indicating the estimated bloodstream information.

Furthermore, the processing circuitry 180 can execute a color Doppler method, also called a color flow mapping (CFM) method, by the Doppler processing function 182. According to the CFM method, transmission and reception of ultrasound is performed multiple times on multiple scanning lines. According to the CFM method, by applying a moving target indicator (MIT) filter to a same-positioned data column, signals (clutter signals) originating from a static tissue or slow-moving tissue are inhibited to extract signals originating from a bloodstream. Furthermore, according to the CFM method, bloodstream information such as a bloodstream velocity, bloodstream dispersion, and bloodstream power is estimated using the extracted flow signals. The image generation function 183 to be described later generates ultrasound image data (color Doppler image data) in which a distribution of the estimated bloodstream information is two-dimensionally displayed in color. Hereinafter, a mode of the ultrasound diagnostic apparatus using the color Doppler method will be referred to as a bloodstream visualization mode. Displaying in color is to display a distribution of bloodstream information by associating it with a predetermined color code, and includes a gray scale.

There are various types of bloodstream visualization modes depending on the desired clinical information. In general, there are a bloodstream visualization mode for velocity display in which a direction of bloodstream and an average velocity of bloodstream are visible, and a bloodstream visualization mode for power display in which a power of a bloodstream signal is visible.

The bloodstream visualization mode for velocity display is a mode in which a color corresponding to the Doppler shift frequency is displayed in accordance with the direction of bloodstream and the average velocity of bloodstream. For example, in the bloodstream visualization mode for velocity display, as directions of flow, an oncoming flow is expressed as a red color while a receding flow is expressed as a blue color, and a difference in velocity between them is expressed by a difference in hue. The bloodstream visualization mode for velocity display may be referred to as a color Doppler mode or a color Doppler imaging (CDI) mode.

The bloodstream visualization mode for power display is, for example, a mode in which a power of a bloodstream signal is expressed by a variation in hue of a red color, the brightness of color (lightness), or the saturation of color. The bloodstream visualization mode for power display may be referred to as power Doppler (PD) mode. The bloodstream visualization mode for power display may be referred to as a high-sensitivity bloodstream visualization mode because it can depict a bloodstream with higher sensitivity than the bloodstream visualization mode for velocity display.

The image generation function 183 is a function for generating B-mode image data based on data generated by the B-mode processing function 181. For example, in the image generation function 183, the processing circuitry 180 converts (scan-converts) a scanning line signal sequence of ultrasound scanning into, for example, a scanning line signal sequence in a video format representatively used by a television, etc., to generate image data for display (display image data). Specifically, the processing circuitry 180 executes RAW-pixel conversion relative to B-mode RAW data stored in the RAW data memory, for example, executes coordinate conversion corresponding to the ultrasound scan state by the ultrasound probe 101, to generate two-dimensional B-mode image data (also referred to as ultrasound image data) constituted by pixels. In other words, by the image generation function 183, the processing circuitry 180 generates a plurality of ultrasound images (medical images) respectively corresponding to a plurality of consecutive frames through transmission and reception of ultrasound waves.

The image generation function 183 further includes a function of generating Doppler image data based on data generated by the Doppler processing function 182. For example, the image generation function 183 performs a RAW-pixel conversion on the Doppler raw data stored in the raw data memory so as to generate Doppler image data in which bloodstream information is visualized. The Doppler image data is average velocity image data, dispersion image data, or power image data, or image data obtained by a combination thereof. The processing circuitry 180 generates, as Doppler image data, color Doppler image data indicating bloodstream information with colors and gray-scale Doppler image data indicating a piece of bloodstream information as waveforms with a gray scale. The color Doppler image data is generated at the time of execution of the bloodstream visualization mode described above.

The decision function 184 decides whether or not a change has been made to a depth of field.

With respect to a scanning line in which a change has been made to the depth of field, the change function 185 makes a change to a pulse repetition frequency (PRF) in accordance with the depth of field after the change.

The setting function 186 sets a new transmission voltage based on the PRF after the change.

The display control function 187 is a function of causing a display serving as the output device 103 to display images based on various types of ultrasound image data generated by the image generation function 183. Specifically, for example, by the display control function 187, the processing circuitry 180 controls the display of an image based on the B-mode image data, the Doppler image data, or image data including both generated by the image generation function 183.

More specifically, by the display control function 187, the processing circuitry 180 performs, for example, conversion (scan conversion) of scan line signal sequences from the ultrasound scanning into scan line signal sequences in a video format as represented by televisions or the like, to generate display image data. The processing circuitry 180 may further perform, on this display image data, various types of processing such as processing for the corrections of dynamic range, brightness (luminance), contrast, and a y-curve, as well as processing for RGB conversion. The processing circuitry 180 may also add supplementary information, such as textual information of various parameters, a scale, or a body mark, to the display image data. The processing circuitry 180 may also generate a user interface (graphical user interface: GUI) to allow the operator to input various instructions through the input device, and cause the display to display the GUI.

The system control function 188 is a function of integrally controlling the overall operations of the ultrasound diagnostic apparatus 1.

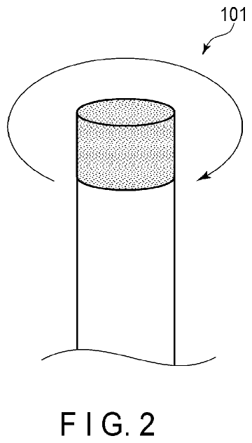

Next, a shape example of the ultrasound probe 101 is shown in FIG. 2.

The ultrasound probe 101 according to the present embodiment is a radial probe, and ultrasound vibrators are arranged 360 degrees around the circumferential direction of the distal end of the ultrasound probe 101 in a columnar shape. This enables a 360-degree scan. Meanwhile, the ultrasound probe 101 may be a mechanical radial probe in which the ultrasound vibrators are not arranged 360 degrees around the probe, but instead are arranged partially around the probe and are rotated circumferentially by a motor to achieve a 360-degree scan. The ultrasound probe 101 is not limited to the radial probe but may be a linear probe. That is, the processing according to the present embodiment is also applicable to the case in which a depth of field is partially changed in the linear probe, in a similar manner to the radial probe.

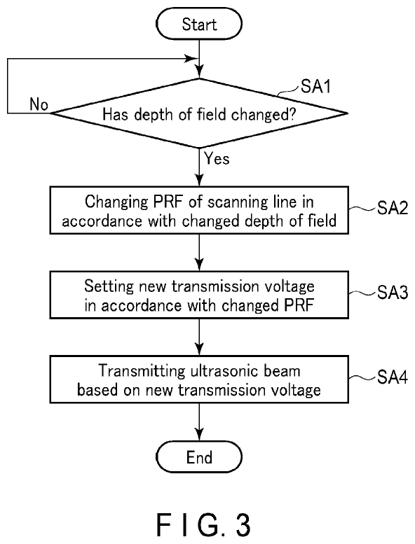

Next, an operation example of the probe control apparatus according to the present embodiment will be described with reference to the flowchart of FIG. 3. Herein, it is assumed that an ultrasound scan is already being performed.

In step SA1, with respect to some of the scanning lines, the processing circuitry 180 decides, through the decision function 184, whether or not a change has been made to a depth of field. For example, in the case where there has been an input for changing a depth of field in a region by a slider switch or a trackball through the input interface 150 by a user's instruction, the processing circuitry 180 can decide that a change has been made to the depth of field. In the case where a change has been made to the depth of field, the processing proceeds to step SA2. In the case where no change has been made to the depth of field, the processing returns to step SA1, and the processing therefrom is repeated until a change is made to the depth of field.

In step SA2, the processing circuitry 180 makes a change, by the change function 185, to a PRF of a scanning line in a region in which the change has been made, in accordance with the depth of field after the change. Specifically, as the depth of field becomes deeper, the transmission interval needs to become longer accordingly, that is, a period during which an echo signal is acquired needs to become longer. Thus, the PRF is lowered. Therefore, the processing circuitry 180 may make a change, by the change function 185, to a PRF in accordance with the depth of field after the change.

In step SA3, the processing circuitry 180 sets, by the setting function 186, a new transmission voltage based on the PRF after the change. Herein, the amount of heat caused by transmission of the transmission voltage and ultrasound beam can be expressed by a relationship determined by Expression (1).

$$Amount of Heat = PRF \times ( Transmission Voltage \times Transmission Voltage ) \qquad ( 1 )$$

The amount of heat caused by the transmission of the ultrasound beam includes, for example, a surface temperature of the ultrasound probe 101 and heat generation of a living body due to the influence of sound power.

It is understood from Expression (1) that the amount of heat can be further suppressed as the PRF becomes lower, that is, the transmission interval becomes longer. Therefore, the lower the PRF, the higher the transmission voltage can be set. On the other hand, the amount of heat is prone to increase as the PRF becomes higher, that is, the transmission interval becomes shorter. This requires the transmission voltage to be set low. The processing circuitry 180 may set, by the setting function 186, a new transmission voltage based on the relationship described above in such a manner that heat generation caused by the transmission of the ultrasound beam becomes equal to or less than an allowable value. Specifically, the new transmission voltage can be calculated by, for example, Expression (2).

$$Transmission Voltage = Transmission Voltage with Reference PRF \times √ ( Reference PRF / PRF ) \qquad ( 2 )$$

In step SA4, the processing circuitry 180 controls, by the system control function 188, the ultrasound transmission circuit 110 and the ultrasound probe 101 in such a manner as to transmit the ultrasound beam with the new transmission voltage.

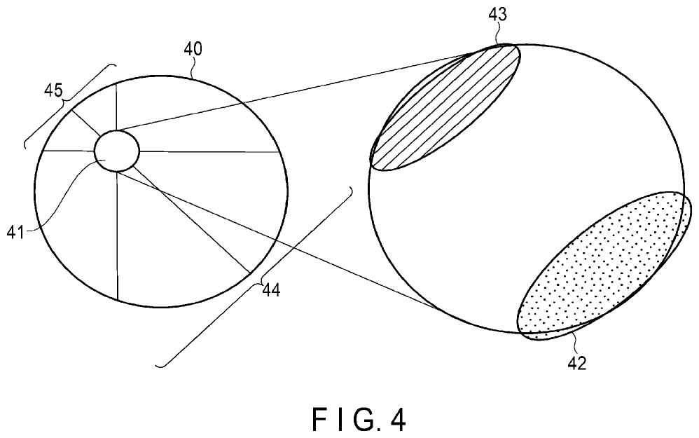

Next, a change state of the depth of field according to the present embodiment will be described with reference to the conceptual diagram of FIG. 4.

FIG. 4 shows a conceptual diagram of the ultrasound image 40 captured with the ultrasound probe 101, which is a radial probe. Furthermore, a probe region 41 in which the ultrasound probe 101 is present can be grasped on the ultrasound image 40.

Herein, in the ultrasound image 40, the depth of field of the scanning lines in the ultrasound probe 101 is deep in the lower right direction and is shallow in the upper left direction. That is, the ultrasound image 40 is not an ultrasound image extending in a concentric circle from the probe region 41 of the ultrasound probe 101 but an ultrasound image in a state in which the depth of field varies depending on the scanning line.

In such a case, in view of the enlarged view of the probe region 41 (the right part of FIG. 4), a PRF can be set higher in a scanning line region 43 corresponding to the scanning lines with the shallow depth of field than a scanning line region 42 corresponding to the scanning lines with the deep depth of field. Thus, the amount of heat is increased. Accordingly, for example, the ultrasound probe 101 may be provided with a temperature sensor configured to measure a surface temperature of a probe and the processing circuitry 180 may set, by the setting function 186, a new transmission voltage for the scanning lines belonging to the scanning line region 42 or the scanning line region 43 in such a manner that the surface temperature of the probe acquired by the temperature sensor becomes equal to or less than an allowable value.

According to the present embodiment described above, with respect to a scanning line in which a change has been made to a depth of field, a change is made to a PRF in accordance with the depth of field after the change, and a new transmission voltage is set based on the PRF after the change. This can secure a desired depth of field while considering heat generation in such a manner that the heat generation becomes equal to or less than an allowable value.

Herein, the term “processor” used in the above explanation means, for example, a circuit such as a central processing unit (CPU), a graphics processing unit (GPU), an application-specific integrated circuit (ASIC), or a programmable logic device (e.g., a simple programmable logic device (SPLD), a complex programmable logic device (CPLD), or a field-programmable gate array (FPGA)). If the processor is, for example, a CPU, the processor reads and executes programs stored in storage circuit to implement the functions. If the processor is, for example, an ASIC, the functions are directly incorporated into the circuit of the processor as a logic circuit, instead of the programs being stored in the storage circuit. Each processor in the present embodiment is not limited to a single circuit-type processor, and multiple independent circuits may be combined and integrated as a single processor to realize the intended functions. Furthermore, the functions may be implemented by a single processor into which multiple components shown in the drawings are incorporated.

Furthermore, the functions described in connection with the above embodiment may be implemented, for example, by installing a program for executing the processing in a computer, such as a workstation, etc., and expanding the program in a memory. The program that causes the computer to execute the processing can be stored and distributed by means of a storage medium, such as a magnetic disk (a hard disk, etc.), an optical disk (CD-ROM, DVD, etc.), and a semiconductor memory.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A probe control apparatus comprising processing circuitry configured to:
with respect to a scanning line in which a change has been made to a depth of field, make a change to a pulse repetition frequency in accordance with the depth of field after the change; and
set a new transmission voltage based on the pulse repetition frequency after the change.

2. The probe control apparatus according to claim 1, wherein the processing circuitry sets, as the new transmission voltage, a value obtained by multiplying a transmission voltage with the pulse repetition frequency before the change by a square root of a ratio of the pulse repetition frequency before the change to the pulse repetition frequency after the change.

3. The probe control apparatus according to claim 1, wherein the processing circuitry sets the new transmission voltage in such a manner that heat generation caused by transmission of an ultrasound beam becomes equal to or less than an allowable value.

4. A non-transitory computer readable medium including computer executable instructions, wherein the instructions, when executed by a processor, cause the processor to perform a method comprising:
with respect to a scanning line in which a change has been made to a depth of field, making a change to a pulse repetition frequency in accordance with the depth of field after the change; and
setting a new transmission voltage based on the pulse repetition frequency after the change.

5. The non-transitory computer readable medium according to claim 4, the method further comprising setting, as the new transmission voltage, a value obtained by multiplying a transmission voltage with the pulse repetition frequency before the change by a square root of a ratio of the pulse repetition frequency before the change to the pulse repetition frequency after the change.

6. The non-transitory computer readable medium according to claim 4, the method further comprising setting the new transmission voltage in such a manner that heat generation caused by transmission of an ultrasound beam becomes equal to or less than an allowable value.

7. An ultrasound diagnostic apparatus comprising:
an ultrasound probe; and
processing circuitry configured to:
decide whether or not a change has been made to a depth of field;
with respect to a scanning line in which a change has been made to the depth of field, make a change to a pulse repetition frequency in accordance with the depth of field after the change;
set a new transmission voltage based on the pulse repetition frequency after the change; and
cause the ultrasound probe to transmit an ultrasound beam in accordance with the new transmission voltage.

8. The ultrasound diagnostic apparatus according to claim 7, wherein the ultrasound probe is a radial probe.
